(19) United States
(12) Patent Application Publication
(10) Pub. No.: US 2008/0138792 A1
(43) Pub. Date: Jun. 12, 2008

(54) VASODILATOR ELUTING MEDICAL DEVICES WITH A SPECIFIC POLYPHOSPHAZENE COATING AND METHODS FOR THEIR MANUFACTURE AND USE

(75) Inventors: Olaf Fritz, Hirschhorn (DE); Ulf Fritz, Hirschhorn (DE); Roman Denk, Weidenstetten (DE); Teresa Wilson, Newnan, GA (US); Ralph E. Gaskins, Atlanta, GA (US)

Correspondence Address: SUTHERLAND ASBILL & BRENNAN LLP, 999 PEACHTREE STREET, N.E., ATLANTA, GA, 30309, US

(73) Assignee: CELONOVA BIOSCIENCES, INC., Newnan, GA (US)

(21) Appl. No.: 11/931,021

(22) Filed: Oct. 31, 2007

Related U.S. Application Data
(63) Continuation-in-part of application No. 11/023,928, filed on Dec. 28, 2004.

(30) Foreign Application Priority Data
Jul. 5, 2002 (DE) 10230190.5
Jul. 4, 2003 (EP) PCT/EP03/07197

(51) Int. Cl. A61L 27/54; C12M 1/00; A01N 1/02; A61L 27/34

(52) U.S. Cl. 435/2; 435/307.1; 523/113; 424/426

(57) ABSTRACT

The present invention is directed to medical devices in which flow is channeled of blood and blood products for the purpose of effecting a chemical exchange to remove desired chemicals from the blood or blood products, and to impart nitric oxide, other smooth muscle relaxant compounds, or other desired chemicals to the blood or blood products to achieve vascular dilatation, reduce adverse reactions, reduce thrombosis, reduce red blood cell injuries, and improve blood handling capabilities. In the present invention, such chemical exchanges occur over semipermeable membranes associated with channeled flow of blood and blood products. Various embodiments of the present invention thus apply to the clinical settings for filters, cannulae, tubing, and blood handling components for dynamic blood handling, filtering, and processing devices including, but not limited to, cardiopulmonary bypass pumps, left ventricular assist devices, artificial hearts, ECMO devices, renal or hepatic hemodialysis systems, and hemofiltration systems.

### CROSS-REFERENCE TO RELATED APPLICATIONS

This application is a continuation-in-part of U.S. patent application Ser. No. 11/023,928, filed Dec. 28, 2004, which claims the benefit of priority of PCT Patent Application No. PCT/EP03/07197, filed Jul. 4, 2003 and German Patent Application No. DE10230190.5, filed Jul. 5, 2002, the entire disclosures of which are incorporated herein by reference.

### BACKGROUND OF THE INVENTION

The present invention is directed to medical devices including dynamic blood handling, filtering, and processing devices that comprise a specific polyphosphazene and a capability of releasing nitric oxide or other smooth muscle relaxant compounds into blood to achieve vascular dilatation, reduce adverse reactions, reduce thrombosis, reduce red blood cell injuries, and improve blood handling capabilities. The medical devices of the present invention include, but are not limited to, such devices as cardiopulmonary bypass pumps, renal dialysis systems, hemofiltration systems, ECMO machines, left ventricular assist devices, and artificial hearts, and related components of such machines and systems.

Nitric oxide (NO) is one of the few gaseous biological signaling molecules known. It is a key biological messenger, playing a role in a variety of biological processes. Nitric oxide, also known as the ‘endothelium-derived relaxing factor’, or ‘EDRF’, is biosynthesized from arginine and oxygen by various nitric oxide synthase (NOS) enzymes and by reduction of inorganic nitrate. The endothelial cells that line blood vessels use nitric oxide to signal the surrounding smooth muscle to relax, thus dilating the artery and increasing blood flow. The production of nitric oxide is elevated in populations living at high-altitudes, which helps these people avoid hypoxia. Effects include blood vessel dilatation, and neurotransmission. Nitroglycerin and amyl nitrite serve as vasodilators because they are converted to nitric oxide in the body.

Phosphodiesterase type 5 inhibitors, often shortened to PDE5 inhibitors. are a class of drugs used to block the degradative action of phosphodiesterase type 5 on cyclic GMP in the smooth muscle cells lining blood vessels. NO activates the enzyme guanylate cyclase which results in increased levels of cyclic guanosine monophosphate (cGMP), leading to smooth muscle relaxation in blood vessels. PDE5 inhibitors inhibit the degradation of cGMP by phosphodiesterase type 5 (PDE5).

Nitric oxide is also generated by macrophages and neutrophils as part of the human immune response. Nitric oxide is toxic to bacteria and other human pathogens. In response, however, many bacterial pathogens have evolved mechanisms for nitric oxide resistance.

A biologically important reaction of nitric oxide is S-nitrosylation, the conversion of thiol groups, including cysteine residues in proteins, to form S-nitrosothiols (RSNOs). S-Nitrosylation is a mechanism for dynamic, post-translational regulation of most or all major classes of protein.

Nitroglycerine or glyceryl trinitrate (GTN) has been used to treat angina and heart failure since at least 1880. Despite this, the mechanism of nitric oxide (NO) generation from GTN and the metabolic consequences of this bioactivation are still not entirely understood.

GTN is a pro-drug which must first be denitrated to produce the active metabolite NO. Nitrates which undergo denitration within the body to produce NO are called nitrovasodilators and their denitration occurs via a variety of mechanisms. The mechanism by which nitrates produce NO is widely disputed. Some believe that nitrates produce NO by reacting with sulfhydryl groups, while others believe that enzymes such as glutathione S-transferases, cytochrome P450 (CYP), and xanthine oxidoreductase are the primary source of GTN bioactivation. In recent years a great deal of evidence has been produced which supports the belief that clinically relevant denitration of GTN to produce 1,2-glyceryl dinitrate (GDN) and NO is catalyzed by mitochondrial aldehyde dehydrogenase (mtALDH). NO is a potent activator of guanylyl cyclase (GC) by heme-dependent mechanisms; this activation results in cGMP formation from guanosine triphosphate (GTP). Thus, NO increases the level of cGMP within the cell.

GTP is more useful in preventing angina attacks than reversing them once they have commenced. Patches of glyceryl trinitrate with long activity duration are commercially available. It may also be given as a sublingual dose in the form of a tablet placed under the tongue or a spray into the mouth for the treatment of an angina attack.

Long acting Nitrates can be more useful as they are generally more effective and stable in the short term. GTP is also used to help provoke a vasovagal syncope attack while having a tilt table test which will then give more accurate results.

It would also be desirable to therapeutically increase the nitrous oxide content in blood in vivo in anatomic areas for treatment for diseases or pathologic conditions in which localized or systemic vasodilatation is compromised.

It would further be desirable to be able to therapeutically increase the nitrous oxide content in blood during in vivo procedures such as renal dialysis, autotransfusion, extracorporeal membrane oxygenation (ECMO), left ventricular assist devices, artificial hearts, and cardiopulmonary bypass, in which blood is being removed from a patient's body, circulated through pumping or filtering devices, and returned to the patient.

It would further be desirable to be able to therapeutically increase the nitrous oxide content in blood during in vivo procedures such as extracorporeal membrane oxygenation (ECMO), left ventricular assist devices, artificial hearts, and cardiopulmonary bypass, in which blood is being removed from a patient's body, circulated through pumping or filtering devices, and then returned to the patient.

### BRIEF SUMMARY OF THE INVENTION

The invention includes a coating for medical devices for use in therapeutic settings where it is desirable to have such devices release nitric oxide or other smooth muscle relaxant drugs into blood or into an anatomic space such as a blood vessel, pancreatic duct, bile duct, tear duct, urethra, ureter, esophagus, intestine, penis, or other anatomic structure whose size is controlled by the action of smooth muscle.

The present invention includes medical devices and component coating for medical devices for use in filters, cannulae, tubing, and blood handling components for dynamic blood handling, filtering, and processing devices such as cardiopulmonary bypass pumps, left ventricular assist devices, artificial hearts, and ECMO devices where it is desirable to have such devices release nitric oxide or other smooth muscle relaxant drugs into blood or other organs where cardiopulmonary function is controlled by the action of smooth muscle.

The present invention further includes medical devices and component coatings for medical devices for use in filters, cannulae, tubing, and blood handling components for dynamic blood handling, filtering, and processing devices such as renal hemodialysis, hemofiltration, and hepatic hemodialysis.

The medical devices of the present invention further comprise poly[bis(trifluoroethoxy)phosphazene] and/or a derivative thereof and one or more smooth muscle relaxant active agents. Poly[(bistrifluorethoxy)phosphazene] has antibacterial and anti-inflammatory properties and inhibits the accumulation of thrombocytes.

Further described herein is a method of delivering an active agent capable of eluting nitric oxide or other smooth muscle relaxants from within a specific polyphosphazene coating into biologic tissue, blood, or serum, or into a space within a medical device comprising the coating as therapeutically desirable.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments that are presently preferred. It should be understood, however, that the invention is not limited to the precise arrangements and instrumentalities shown.

In the drawings:

FIG. 1 shows a schematic cross-sectional representation of an exemplary embodiment of the present invention in which a plurality of tubules are surrounded by interstitial flow space.

FIG. 2 shows a more detailed view of the schematic cross-sectional representation of FIG. 1.

FIG. 3 shows a schematic cross-sectional representation of another exemplary embodiment of the present invention in which a plurality of first tubules interface with a plurality of second tubules to allow chemical exchanges through the semipermeable tubule walls of the first and second tubules.

FIG. 4 shows a more detailed view of the schematic cross-sectional representation of FIG. 3.

### DETAILED DESCRIPTION OF THE INVENTION

The present invention may be understood more readily by reference to the following detailed description of the preferred embodiments of the invention and the examples included herein. However, before the preferred embodiments of the devices and methods according to the present invention are disclosed and described, it is to be understood that this invention is not limited to the exemplary embodiments described within this disclosure, and the numerous modifications and variations therein that will be apparent to those skilled in the art remain within the scope of the invention disclosed herein. It is also to be understood that the terminology used herein is for the purpose of describing specific embodiments only and is not intended to be limiting.

Unless otherwise noted, the terms used herein are to be understood according to conventional usage by those of ordinary skill in the relevant art. In addition to the definitions of terms provided below, it is to be understood that as used in the specification and in the claims, “a” or “an” can mean one or more, depending upon the context in which it is used.

Described herein are medical devices comprising poly[bis(trifluoroethoxy)phosphazene] and/or a derivative thereof and one or smooth muscle relaxant active agents capable of release into the biologic tissues, blood, or serum of a patient, or into a space within a medical device comprising the coating upon deployment or use of the devices.

Further described herein are methods for the manufacture and use of medical devices comprising poly[bis(trifluoroethoxy)phosphazene] and/or a derivative thereof and one or more nitrogen compounds or other smooth muscle relaxant active agents capable of release during storage of biological or pharmaceutical containment or administration therein, or into the biologic tissues, blood, or serum of a patient, or into a space within a medical device comprising the coating upon deployment or use of the devices.

In certain embodiments of the present invention, medical devices are provided with a polymeric coating comprising poly[bis(trifluoroethoxy) phosphazene] and/or a derivative thereof releasably bonded to compounds capable of producing nitric oxide or other bioactive nitrogen compounds upon release from the polymer.

The present invention further includes methods for the manufacture and use of medical devices comprising a polymeric coating comprising poly[bis(trifluoroethoxy)phosphazene] and/or a derivative thereof releasably bonded to compounds capable of producing nitric oxide or other bioactive nitrogen compounds upon release from the polymer.

As described herein, the polymer poly[bis(2,2,2-trifluoroethoxy)phosphazene] or derivatives thereof have chemical and biological qualities that distinguish this polymer from other know polymers in general, and from other know polyphosphazenes in particular. In one aspect of this invention, the polyphosphazene is poly[bis(2,2,2-trifluoroethoxy) phosphazene] or derivatives thereof, such as other alkoxide, halogenated alkoxide, or fluorinated alkoxide substituted analogs thereof. The preferred poly[bis(trifluoroethoxy)phosphazene] polymer is made up of repeating monomers represented by the formula (I) shown below:

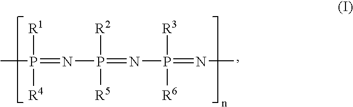

wherein R¹ to R⁶ are all trifluoroethoxy (OCH₂CF₃) groups, and wherein n may vary from at least about 40 to about 100,000, as disclosed herein. Alternatively, one may use derivatives of this polymer in the present invention. The term “derivative” or “derivatives” is meant to refer to polymers made up of monomers having the structure of formula I but where one or more of the R¹ to R⁶ functional group(s) is replaced by a different functional group(s), such as an unsubstituted alkoxide, a halogenated alkoxide, a fluorinated alkoxide, or any combination thereof or where one or more of the R¹ to R⁶ is replaced by any of the other functional group(s) disclosed herein, but where the biological inertness of the polymer is not substantially altered.

In one aspect of the polyphosphazene of formula (I) illustrated above, for example, at least one of the substituents R¹ to R⁶ can be an unsubstituted alkoxy substituent, such as methoxy (OCH₃), ethoxy (OCH₂CH₃) or n-propoxy (OCH₂CH₂CH₃). In another aspect, for example, at least one of the substituents R¹ to R⁶ is an alkoxy group substituted with at least one fluorine atom. Examples of useful fluorine-substituted alkoxy groups R¹ to R⁶ include, but are not limited to OCF₃, OCH₂CF₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂, OCH₂(CF₂)₆CHF₂, OCH₂(CF₂)₇CHF₂, and the like. Thus, while trifluoroethoxy (OCH₂CF₃) groups are preferred, these further exemplary functional groups also may be used alone, in combination with trifluoroethoxy, or in combination with each other. In one aspect, examples of especially useful fluorinated alkoxide functional groups that may be used include, but are not limited to, 2,2,3,3,3-pentafluoropropyloxy (OCH₂CF₂CF₃), 2,2,2,2′,2′,2′-hexafluoroisopropyloxy (OCH(CF₃)₂), 2,2,3,3,4,4,4-heptafluorobutyloxy (OCH₂CF₂CF₂CF₃), 3,3,4,4,5,5,6,6,7,7,8,8,8-tridecafluorooctyloxy (OCH₂(CF₂)₇CF₃), 2,2,3,3,-tetrafluoropropyloxy (OCH₂CF₂CHF₂), 2,2,3,3,4,4-hexafluorobutyloxy (OCH₂CF₂CF₂CHF₂), 3,3,4,4,5,5,6,6,7,7,8,8-dodecafluorooctyloxy (OCH₂(CF₂)₇CHF₂), and the like, including combinations thereof.

Further, in some embodiments, 1% or less of the R¹ to R⁶ groups may be alkenoxy groups, a feature that may assist in crosslinking to provide a more elastomeric phosphazene polymer. In this aspect, alkenoxy groups include, but are not limited to, OCH₂CH═CH₂, OCH₂CH₂CH═CH₂, allylphenoxy groups, and the like, including combinations thereof. Also in formula (I) illustrated herein, the residues R¹ to R⁶ are each independently variable and therefore can be the same or different.

By indicating that n can be as large as ∞ in formula I, it is intended to specify values of n that encompass polyphosphazene polymers that can have an average molecular weight of up to about 75 million Daltons. For example, in one aspect, n can vary from at least about 40 to about 100,000. In another aspect, by indicating that n can be as large as ∞ in formula I, it is intended to specify values of n from about 4,000 to about 50,000, more preferably, n is about 7,000 to about 40,000 and most preferably n is about 13,000 to about 30,000.

In another aspect of this invention, the polymer used to prepare the polymers disclosed herein has a molecular weight based on the above formula, which can be a molecular weight of at least about 70,000 g/mol, more preferably at least about 1,000,000 g/mol, and still more preferably a molecular weight of at least about 3×10⁶ g/mol to about 20×10⁶ g/mol. Most preferred are polymers having molecular weights of at least about 10,000,000 g/mol.

In a further aspect of the polyphosphazene formula (I) illustrated herein, n is 2 to ∞, and R¹ to R⁶ are groups which are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof, or heteroaryl comprising one or more heteroatoms selected from nitrogen, oxygen, sulfur, phosphorus, or a combination thereof. In this aspect of formula (I), the pendant side groups or moieties (also termed “residues”) R¹ to R⁶ are each independently variable and therefore can be the same or different. Further, R¹ to R⁶ can be substituted or unsubstituted. The alkyl groups or moieties within the alkoxy, alkylsulphonyl, dialkylamino, and other alkyl-containing groups can be, for example, straight or branched chain alkyl groups having from 1 to 20 carbon atoms, typically from 1 to 12 carbon atoms, it being possible for the alkyl groups to be further substituted, for example, by at least one halogen atom, such as a fluorine atom or other functional group such as those noted for the R¹ to R⁶ groups above. By specifying alkyl groups such as propyl or butyl, it is intended to encompass any isomer of the particular alkyl group.

In one aspect, examples of alkoxy groups include, but are not limited to, methoxy, ethoxy, propoxy, and butoxy groups, and the like, which can also be further substituted. For example the alkoxy group can be substituted by at least one fluorine atom, with 2,2,2-trifluoroethoxy constituting a useful alkoxy group. In another aspect, one or more of the alkoxy groups contains at least one fluorine atom. Further, the alkoxy group can contain at least two fluorine atoms or the alkoxy group can contain three fluorine atoms. For example, the polyphosphazene that is combined with the silicone can be poly[bis(2,2,2-trifluoroethoxy)phosphazene]. Alkoxy groups of the polymer can also be combinations of the aforementioned embodiments wherein one or more fluorine atoms are present on the polyphosphazene in combination with other groups or atoms.

Examples of alkylsulphonyl substituents include, but are not limited to, methylsulphonyl, ethylsulphonyl, propylsulphonyl, and butylsulphonyl groups. Examples of dialkylamino substituents include, but are not limited to, dimethyl-, diethyl-, dipropyl-, and dibutylamino groups. Again, by specifying alkyl groups such as propyl or butyl, it is intended to encompass any isomer of the particular alkyl group.

Exemplary aryloxy groups include, for example, compounds having one or more aromatic ring systems having at least one oxygen atom, non-oxygenated atom, and/or rings having alkoxy substituents, it being possible for the aryl group to be substituted for example by at least one alkyl or alkoxy substituent defined above. Examples of aryloxy groups include, but are not limited to, phenoxy and naphthoxy groups, and derivatives thereof including, for example, substituted phenoxy and naphthoxy groups.

The heterocycloalkyl group can be, for example, a ring system which contains from 3 to atoms, at least one ring atom being a nitrogen, oxygen, sulfur, phosphorus, or any combination of these heteroatoms. The heterocycloalkyl group can be substituted, for example, by at least one alkyl or alkoxy substituent as defined above. Examples of heterocycloalkyl groups include, but are not limited to, piperidinyl, piperazinyl, pyrrolidinyl, and morpholinyl groups, and substituted analogs thereof.

The heteroaryl group can be, for example, a compound having one or more aromatic ring systems, at least one ring atom being a nitrogen, an oxygen, a sulfur, a phosphorus, or any combination of these heteroatoms. The heteroaryl group can be substituted for example by at least one alkyl or alkoxy substituent defined above. Examples of heteroaryl groups include, but are not limited to, imidazolyl, thiophene, furane, oxazolyl, pyrrolyl, pyridinyl, pyridinoyl, isoquinolinyl, and quinolinyl groups, and derivatives thereof such as substituted groups.

As disclosed herein, smooth muscle relaxant active agents or compounds capable of producing nitric oxide or other bioactive nitrogen compounds upon release of the present invention further comprise diazeniumdiolates, sodium nitroprusside, molsidomine, nitrate esters, the S-nitrosothiol family, L-arginine, nitric oxide-nucleophile complexes, glyceryl trinitrate, nitric oxide-primary amine complexes, and related compounds, esters, amines, or other compositions thereof. Smooth muscle relaxant active agents or compounds capable of producing nitric oxide or other bioactive nitrogen compounds upon release of the present invention may further comprise any other inorganic or organic composition capable of forming nitric oxide upon chemical degradation.

In various embodiments of the present invention, the release of smooth muscle relaxant active agents or compounds capable of producing nitric oxide or other bioactive nitrogen compounds may be a spontaneous degradation of the bonding of such agents or compounds to the polymer or substrate. Alternatively, the release of smooth muscle relaxant active agents or compounds capable of producing nitric oxide or other bioactive nitrogen compounds may be controlled by the regulation of pH or other similar chemical or physical factors that might break amide, ether or ester bonds and thus facilitate release.

In certain preferred embodiments of the present invention, diazeniumdiolates are incorporated into blood-insoluble polyphosphazene polymers that generate molecular NO at their surfaces. In other preferred embodiments of the present invention, diazeniumdiolates may be applied to a substrate surface of a medical device as an intermediate coating, which is then coated with the preferred poly[bis(trifluoroethoxy)phosphazene] polymer of the present invention. In yet other preferred embodiments of the present invention, a substrate surface of a medical device may receive a first coating with the preferred poly[bis(trifluoroethoxy)phosphazene] polymer of the present invention, followed by an intermediate coating of diazeniumdiolates, followed by a second coating of the poly[bis(trifluoroethoxy)phosphazene] polymer as described herein. In such embodiments with a first and second coating of the poly[bis(trifluoroethoxy)phosphazene] polymer, the first and second coatings may each be bioabsorbable or non-bioabsorbable.

Diazeniumdiolates are now available with a range of half-lives for spontaneous NO release. The ability of the diazeniumdiolates to generate copious NO at rates that vary widely is largely independent of metabolic or medium effects.

Other preferred embodiments of the present invention may use other nitric oxide-eluting or other smooth muscle relaxant compounds, including, but not limited to sodium nitroprusside, molsidomine, nitrate esters, the S-nitrosothiol family, L-arginine, nitric oxide-nucleophile complexes, glyceryl trinitrate, nitric oxide-primary amine complexes, and related compounds. In such various embodiments of the present invention, the nitric oxide-eluting or other smooth muscle relaxant compounds may be incorporated into non-bioabsorbable polyphosphazene polymers that generate molecular NO at their surfaces. In other preferred embodiments of the present invention, nitric oxide-eluting or other smooth muscle relaxant compounds may be applied to a substrate surface of a medical device as an intermediate coating, which is then coated with the preferred poly[bis(trifluoroethoxy)phosphazene] polymer of the present invention. In yet other preferred embodiments of the present invention, a substrate surface of a medical device may receive a first coating with the preferred poly[bis(trifluoroethoxy)phosphazene] polymer of the present invention, followed by an intermediate coating of nitric oxide-eluting or other smooth muscle relaxant compounds, followed by a second coating of the poly[bis(trifluoroethoxy)phosphazene] polymer as described herein. In such embodiments with a first and second coating of the poly[bis(trifluoroethoxy)phosphazene] polymer, the first and second coatings may each be bioabsorbable or non-bioabsorbable.

The medical devices disclosed herein may comprise the poly[bis(trifluoroethoxy)phosphazene] polymer represented by formula (I) in various forms: as a coating, as a film, or as a solid structural component. When used as a coating or film in embodiments of the present invention, the poly[bis(trifluoroethoxy)phosphazene] polymer may be provided in varying degrees of porosity, or as a solid surface. Coatings of medical devices of the present invention may be accomplished by any known coating process, including but not limited to dip coating, spray coating, spin coating, brush coating, electrostatic coating, electroplating, electron beam-physical vapor deposition, and other coating technologies.

Similarly, the poly[bis(trifluoroethoxy)phosphazene] polymer may be provided as either a bioabsorbable or non-bioabsorbable form as most appropriate in various embodiments of the present invention. In various embodiments of the present invention, two or more coatings of the poly[bis(trifluoroethoxy)phosphazene] polymer may be applied to the surface of a medical device, and the two or more coatings of the poly[bis(trifluoroethoxy)phosphazene] polymer may be independently provided as bioabsorbable or non-bioabsorbable.

In one embodiment of the present invention an adhesion promoter may be provided in a layer between the surface of the substrate and the polymeric coating.

In exemplary embodiments of the present invention, the adhesion promoter is an organosilicon compound, preferably an amino-terminated silane or a compound based on an aminosilane, or an alkylphosphonic acid. Aminopropyltrimethoxysilane is a preferred adhesion promoter according to the present invention.

In various exemplary embodiments of the present invention, the adhesion promoter particularly improves the adhesion of the coating to the surface of the implant material through coupling of the adhesion promoter to the surface of the implant material, through, for instance, ionic and/or covalent bonds, and through further coupling of the adhesion promoter to reactive components, particularly to the antithrombogenic polymer of the coating, through, for instance, ionic and/or covalent bonds.

For most cardiothoracic operations such as coronary artery bypass grafting, the cardiopulmonary bypass is performed using a heart-lung machine (or cardiopulmonary bypass machine). The heart-lung machine serves to replace the work of the heart during the open bypass surgery. The machine replaces both the heart's pumping action, and adds oxygen to the blood. Since the heart is stopped during the operation, this permits the surgeon to operate on a bloodless, stationary heart.

One component of the heart-lung machine is the oxygenator. The oxygenator component serves as the lung, and is designed to expose the blood to oxygen. It is disposable, and contains about 2-4 m² of a membrane permeable to gas but impermeable to blood, in the form of hollow fibers. Blood flows on the outside of the hollow fibers, while oxygen flows in the opposite direction on the inside of the fibers. As the blood passes through the oxygenator, the blood comes into intimate contact with the fine surfaces of the device itself. Oxygen gas is delivered to the interface between the blood and the device, permitting the blood cells to absorb oxygen molecules directly.

Operations which involve uncoated oxygenators require a high dose of systemic heparinization. There are a number of side effects associated with this. The primary side effect can be post-operative hemorrhage. Systemic heparin does not completely prevent clotting or the activation of complement, neutrophils, and monocytes, which are the principal mediators of the inflammatory response. This response produces a wide range of cytotoxins, and cell-signaling proteins that circulate throughout the patient's body during surgery and disrupt homeostasis. Both the thrombotic and inflammatory responses produce thousands of microembolic particles. Microparticles obstruct arterioles that supply small nests of cells throughout the body and, together with cytotoxins, damage organs and tissues and temporarily disturb organ function. Additionally, bare oxygenators are often associated with neurological symptoms following perfusion. Physicians refer to such temporary neurological deficits as “pumphead syndrome.” The addition of nitric oxide during blood oxygenation is desirable for patients, surgeons, and perfusionists.

Renal and hepatic dialysis procedures are similar to cardiopulmonary bypass in that blood is removed in vivo to undergo external chemical exchanges using semipermeable membrane technologies, and then is returned directly to the patient.

In medicine, renal dialysis is primarily used to provide an artificial replacement for lost kidney function (renal replacement therapy) due to renal failure. Dialysis may be used for very sick patients who have suddenly but temporarily, lost their kidney function (acute renal failure) or for quite stable patients who have permanently lost their kidney function (end stage renal failure). When healthy, the kidneys maintain the body's internal equilibrium of water and minerals (sodium, potassium, chloride, calcium, phosphorus, magnesium, sulfate) and the kidneys remove from the blood the daily metabolic load of fixed hydrogen ions. The kidneys also function as a part of the endocrine system producing erythropoietin and 1,25-dihydroxycholecalciferol (calcitriol). Dialysis treatments imperfectly replace some of these functions through the diffusion (waste removal) and convection (fluid removal). Dialysis is an imperfect treatment to replace kidney function because it does not correct the endocrine functions of the kidney.

Renal dialysis works on the principles of the diffusion and osmosis of solutes and fluid across a semipermeable membrane. Blood flows by one side of a semipermeable membrane, and a dialysate or fluid flows by the opposite side. Smaller solutes and fluid pass through the membrane. The blood flows in one direction and the dialysate flows in the opposite. The concentrations of undesired solutes (for example potassium, calcium, and urea) are high in the blood, but low or absent in the dialysis solution and constant replacement of the dialysate ensures that the concentration of undesired solutes is kept low on this side of the membrane. The dialysis solution has levels of minerals like potassium and calcium that are similar to their natural concentration in healthy blood. For another solute, bicarbonate, dialysis solution level is set at a slightly higher level than in normal blood, to encourage diffusion of bicarbonate into the blood, to neutralize the metabolic acidosis that is often present in these patients.

In renal hemodialysis, the patient's blood is pumped through the blood compartment of a dialyzer, exposing it to a semipermeable membrane. The cleansed blood is then returned via the circuit back to the body. Ultrafiltration occurs by increasing the hydrostatic pressure across the dialyzer membrane. This usually is done by applying a negative pressure to the dialysate compartment of the dialyzer. This pressure gradient causes water and dissolved solutes to move from blood to dialysate, and allows removal of several litres of excess fluid during a typical 3 to 5 hour treatment.

The principle of renal hemodialysis is the same as other methods of dialysis; it involves diffusion of solutes across a semipermeable membrane. Hemodialysis utilizes counter current flow, where the dialysate is flowing in the opposite direction to blood flow in the extracorporeal circuit. Counter-current flow maintains the concentration gradient across the membrane at a maximum and increases the efficiency of the dialysis.

Fluid removal (ultrafiltration) is achieved by altering the hydrostatic pressure of the dialysate compartment, causing free water and some dissolved solutes to move across the membrane along a created pressure gradient. The dialysis solution that is used is a sterilized solution of mineral ions. Urea and other waste products, and also, potassium and phosphate, diffuse into the dialysis solution. However, concentrations of sodium and chloride are similar to those of normal plasma to prevent loss. Bicarbonate is added in a higher concentration than plasma to correct blood acidity. A small amount of glucose is also commonly used.

Hemofiltration is a similar treatment to hemodialysis, but it makes use of a different principle. The blood is pumped through a dialyzer or “hemofilter” as in dialysis, but no dialysate is used. A pressure gradient is applied; as a result, water moves across the very permeable membrane rapidly, facilitating the transport of dissolved substances, importantly ones with large molecular weights, which are cleared less well by hemodialysis. Salts and water lost from the blood during this process are replaced with a “substitution fluid” that is infused into the extracorporeal circuit during the treatment. Hemodiafiltration is a term used to describe several methods of combining hemodialysis and hemofiltration in one process.

As in dialysis, in hemofiltration one achieves movement of solutes across a semi-permeable membrane. However, solute movement with hemofiltration is governed by convection rather than by diffusion. With hemofiltration, dialysate is not used. Instead, a positive hydrostatic pressure drives water and solutes across the filter membrane from the blood compartment to the filtrate compartment, from which it is drained. Solutes, both small and large, get dragged through the membrane at a similar rate by the flow of water that has been engendered by the hydrostatic pressure. So convection overcomes the reduced removal rate of larger solutes (due to their slow speed of diffusion) seen in hemodialysis.

An isotonic replacement fluid is added to the blood to replace fluid volume and electrolytes. The replacement fluid must be of high purity, because it is infused directly into the blood line of the extracorporeal circuit. The replacement hemofiltration fluid usually contains lactate or acetate as a bicarbonate-generating base, or bicarbonate itself. Use of lactate can occasionally be problematic in patients with lactic acidosis or with severe liver disease, because in such cases the conversion of lactate to bicarbonate can be impaired. In such patients use of bicarbonate as a base is preferred.

Hemofiltration is sometimes used in combination with hemodialysis, when it is termed hemodiafiltration. Blood is pumped through the blood compartment of a high flux dialyzer, and a high rate of ultrafiltration is used, so there is a high rate of movement of water and solutes from blood to dialysate that must be replaced by substitution fluid that is infused directly into the blood line. However, dialysis solution is also run through the dialysate compartment of the dialyzer. The combination is theoretically useful because it results in good removal of both large and small molecular weight solutes.

Hepatic dialysis is a detoxification treatment for developed for liver failure and has shown promise for patients with hepatorenal syndrome. It is similar to hemodialysis and based on the same principles. Like a bioartificial liver device, it is a form of artificial extracorporeal liver support.

A critical issue of the clinical syndrome in liver failure is the accumulation of toxins not cleared by the failing liver. Based on this hypothesis, the removal of lipophilic, albumin-bound substances such as bilirubin, bile acids, metabolites of aromatic amino acids, medium-chain fatty acids and cytokines should be beneficial to the clinical course of a patient in liver failure. This led to the development of artificial filtration and adsorption devices.

Hepatic hemodialysis is used for renal failure and primarily removes water soluble toxins, however it does not remove toxins bound to albumin that accumulate in liver failure.

Artificial detoxification devices currently under clinical evaluation include the Molecular Adsorbent Recirculating System (MARS®), Single Pass Albumin Dialysis (SPAD®) and the Prometheus® system.

The molecular adsorbents recirculation system (MARS®), developed by Terakin AG of Germany, is the best known extracorporal liver dialysis system and has existed for approximately ten years. It consists of two separate dialysis circuits. The first circuit consists of human serum albumin, is in contact with the patients blood through a semipermeable membrane and has two special filters to clean the albumin after it has absorbed toxins from the patient's blood. The second circuit consists of a hemodialysis machine and is used to clean the albumin in the first circuit, before it is recirculated to the semipermeable membrane in contact with the patient's blood. The MARS® system can remove a number of toxins, including ammonia, bile acids, bilirubin, copper, iron and phenols.

Single pass albumin dialysis (SPAD®) is a simple method of albumin dialysis using standard renal replacement therapy machines without an additional perfusion pump system: The patient's blood flows through a circuit with a high-flux hollow fiber hemodiafilter, identical to that used in the MARS® system. The other side of this membrane is cleansed with an albumin solution in counter-directional flow, which is discarded after passing the filter. Hemodialysis can be performed in the first circuit via the same high-flux hollow fibers.

SPAD®, MARS® and continuous veno-venous hemodiafiltration (CVVHDF) have been compared in vitro with regard to detoxification capacity. SPAD® and CVVHDF showed a significantly greater reduction of ammonia compared with MARS. No significant differences could be observed between SPAD®, MARS® and CVVHDF concerning other water-soluble substances. However, SPAD® enabled a significantly greater bilirubin reduction than MARS®. Bilirubin serves as an important marker substance for albumin-bound (non water-soluble) substances. Concerning the reduction of bile acids no significant differences between SPAD® and MARS® were seen. It was concluded that the detoxification capacity of SPAD® is similar or even higher when compared with the more sophisticated, more complex and hence more expensive MARS®.

The Prometheus® system (Fresenius Medical Care, Bad Homburg, Germany) is a device based on the combination of albumin adsorption with high-flux hemodialysis after selective filtration of the albumin fraction through a specific polysulfon filter (AlbuFlow®). It has been studied in a group of eleven patients with hepatorenal syndrome (acute-on-chronic liver failure and accompanying renal failure). The treatment for two consecutive days for more than four hours significantly improved serum levels of conjugated bilirubin, bile acids, ammonia, cholinesterase, creatinine, urea and blood pH.

Nitric oxide is a naturally occurring and potent anti-platelet agent and enhanced nitric oxide levels may greatly decrease the risk of thrombosis during and after renal or hepatic hemodialysis and/or hemofiltration procedures.

In certain exemplary embodiments of the present invention in which chemicals may be removed from and/or added to transient flowing blood using semipermeable membranes, films, or coatings comprising poly[bis(trifluoroethoxy)phosphazene] and/or a derivative thereof releasably bonded to compounds capable of producing nitric oxide or other bioactive nitrogen compounds upon release from the polymer.

The devices, coatings, and methods of the present invention are directed to situations involving channeled flow of blood and blood products for the purpose of effecting a chemical exchange to remove desired chemicals from the blood or blood products, and to impart other desired chemicals to the blood or blood products. Such chemical exchanges occur over semipermeable membranes associated with such channeled flow of blood and blood products within tubules of the present invention or within interstitial flow spaces within the various dynamic blood handling, filtering, and processing devices of the present invention. Various embodiments of the present invention thus apply to the clinical settings for filters, cannulae, tubing, and blood handling components for dynamic blood handling, filtering, and processing devices including, but not limited to, cardiopulmonary bypass pumps, left ventricular assist devices, artificial hearts, ECMO devices, renal or hepatic hemodialysis systems, and hemofiltration systems.

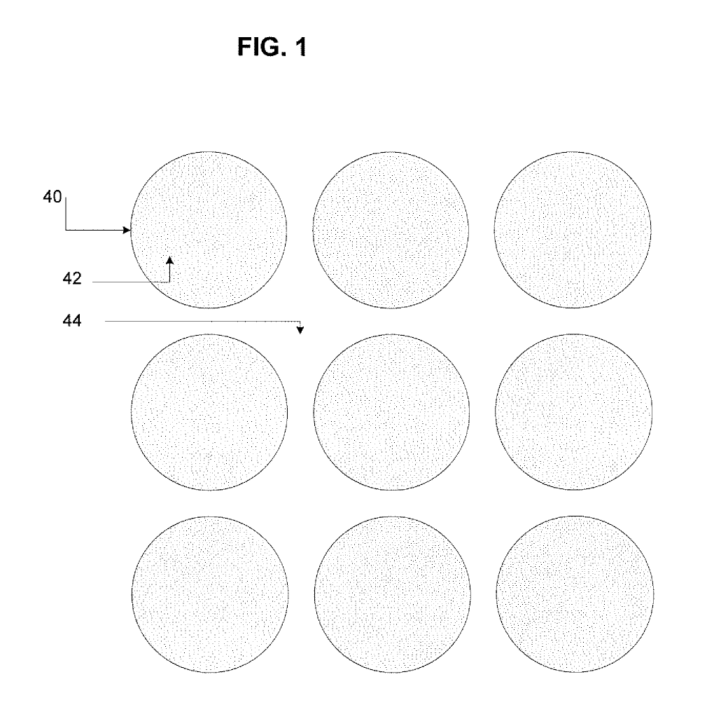

FIG. 1 shows a schematic cross-sectional representation of an exemplary embodiment of the present invention in which a plurality of tubules 40 with lumens 42 are surrounded by interstitial flow space 44. In such an embodiment of the present invention, tubules 40 comprise semipermeable membranes, films, or coatings which may be provided as structural elements or on a semipermeable substrate material, with the semipermeable membranes, films, or coatings comprising poly[bis(trifluoroethoxy)phosphazene] and/or a derivative thereof releasably bonded to compounds capable of producing nitric oxide or other bioactive nitrogen compounds upon release from the polymer.

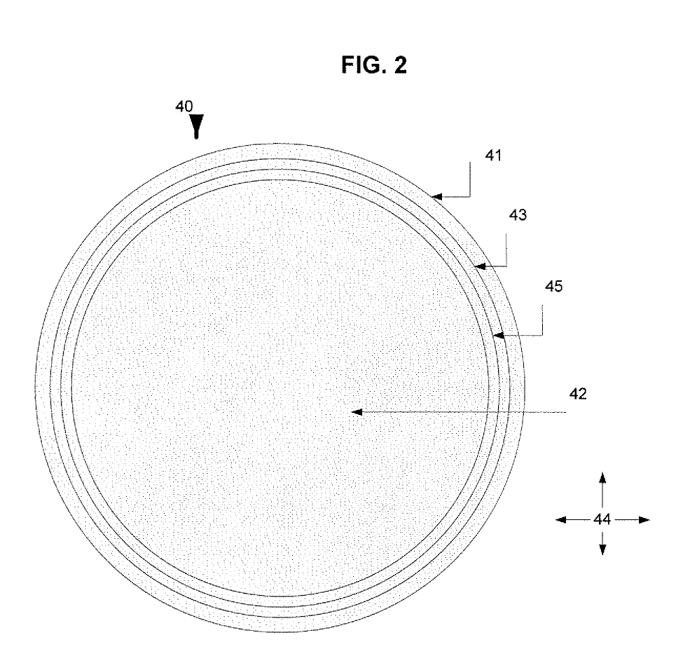

FIG. 2 shows a more detailed view of the structure of the exemplary embodiment shown in FIG. 1. In FIG. 2, tubules 40 comprise a lumen 42, surrounded by an inner semipermeable polymeric coating 45, a semipermeable membranous substrate tubule wall 43, and an outer semipermeable polymeric coating 41.

In the exemplary embodiments of the present invention as shown in FIG. 2, any combination of the inner semipermeable polymeric coating 45, semipermeable membranous substrate tubule wall 43, and outer semipermeable polymeric coating 41 may further comprise reversibly bonded nitric oxide eluting compounds, including any nitrogen compound capable of in vivo breakdown to nitric oxide or other nitrite or nitrate compounds.

The exemplary embodiment of the present invention as shown in FIGS. 1 and 2 may function in several ways in different clinical applications of the present invention. Afferent blood or other fluids may be routed through the lumen 42 of tubules 40, and chemicals such as oxygen may be routed through the interstitial flow space 44, allowing chemical exchanges including the accretion of nitric oxide or other vasoactive nitrogen compounds within the transient blood to occur through the semipermeable inner polymeric coating 45, semipermeable membranous substrate tubule wall 43, and semipermeable outer polymeric coating 41.

Conversely, in other exemplary embodiments of the present invention, afferent blood or other fluids may be routed through interstitial flow space 44, and efferent waste material and other fluids may be accumulated in the lumen 42 of tubules 40, following chemical exchanges through the semipermeable inner polymeric coating 45, semipermeable membranous substrate tubule wall 43, and semipermeable outer polymeric coating 41.

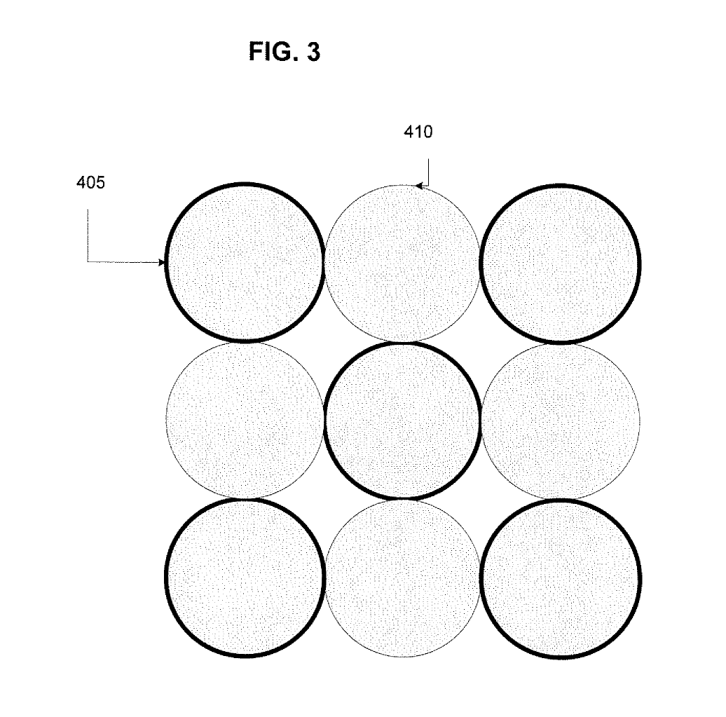

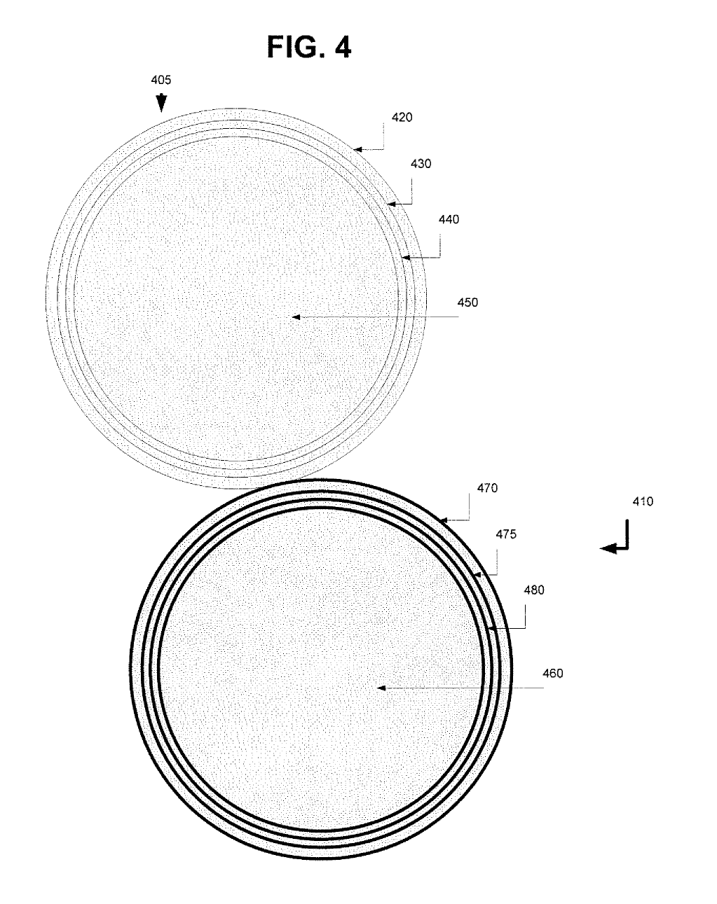

FIGS. 3 and 4 show schematic cross-sectional representations of another exemplary embodiment of the present invention in which a plurality of first tubules interface with a plurality of second tubules to allow chemical exchanges through the semipermeable tubule walls of the first and second tubules.

In FIG. 3, a cross-sectional view is provided for a network of first tubules 405 and second tubules 410. While shown in parallel arrangement in FIG. 3, the first tubules 405 and second tubules 410 may be configured in a mesh or in any other physical arrangement that allows sufficient contact between the first tubules 405 and second tubules 410 to permit the desired chemical exchanges through their respective semipermeable tubule walls in various embodiments of the present invention. Moreover, the present invention includes the possibility for a plurality of types of tubules, so that a given embodiment could involve first, second, third, and fourth tubules, should a four-way division of flow be desirable in given situations. The number of such divisions of tubules in the present invention is unlimited.

FIG. 4 shows a detail of the exemplary embodiment of FIG. 3, in which a first tubule 405 comprises a lumen 450, surrounded by an inner semipermeable polymeric coating 440, a semipermeable membranous substrate tubule wall 430, and an outer semipermeable polymeric coating 420. The first tubule 405 is shown to be in contact with at least one second tubule 410, which comprises a lumen 460, surrounded by an inner semipermeable polymeric coating 480, a semipermeable membranous substrate tubule wall 475, and an outer semipermeable polymeric coating 470.

In the exemplary embodiments of the present invention as shown in FIG. 4, any combination of the inner semipermeable polymeric coating 440, semipermeable membranous substrate tubule wall 430, and outer semipermeable polymeric coating 420 of the first tubule 405 and the inner semipermeable polymeric coating 480, semipermeable membranous substrate tubule wall 475, and outer semipermeable polymeric coating 470 of the second tubule 410 may further comprise reversibly bonded nitric oxide eluting compounds, including any nitrogen compound capable of in vivo breakdown to nitric oxide or other nitrite or nitrate compounds. Blood in transit through the first and or second tubules of FIG. 4 may thus receive an accretion of nitric oxide or other nitrogen-containing vasoactive compounds or other smooth muscle relaxant agents, in addition to other desired chemical exchanges, removals, or additions.

It will be appreciated by those possessing ordinary skill in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A dynamic blood handling or filtration device, comprising:
a. a plurality of tubules, at least some of which comprise semipermeable membranous substrate tubule walls;
b. a specific polyphosphazene component, the polyphosphazene having the formula: 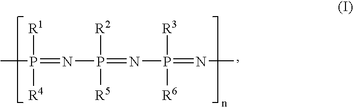
n is 2 to ∞; and
R¹ to R⁶ are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof, or heteroaryl comprising one or more heteroatoms selected from nitrogen, oxygen, sulfur, phosphorus, or a combination thereof.
c. a smooth muscle relaxant active agent.

2. The dynamic blood handling or filtration device according to claim 1, wherein at least one of R¹ to R⁶ is an alkoxy group substituted with at least one fluorine atom.

3. The dynamic blood handling or filtration device according to claim 1, wherein R¹ to R⁶ are selected independently from OCH₃, OCH₂CH₃, OCH₂CH₂CH₃, OCF₃, OCH₂CF₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂, OCH₂(CF₂)₆CHF₂, OCH₂(CF₂)₇CHF₂, OCH₂CH═CH₂, OCH₂CH₂CH═CH₂, or any combination thereof.

4. The dynamic blood handling or filtration device according to claim 1, wherein the polyphosphazene is poly[bis(2,2,2-trifluoroethoxy)]phosphazene or a derivative of poly[bis(2,2,2-trifluoroethoxy)]phosphazene.

5. The dynamic blood handling or filtration device according to claim 1, wherein the polyphosphazene component is at least one coating for the semipermeable membranous substrate tubule wall.

6. The dynamic blood handling or filtration device according to claim 1, wherein the smooth muscle relaxant active agent is releasably bonded to the polyphosphazene component.

7. The dynamic blood handling or filtration device according to claim 1, wherein the smooth muscle relaxant active agent is a compound capable of producing nitric oxide or other bioactive nitrogen compounds upon release from the polyphosphazene component.

8. The dynamic blood handling or filtration device according to claim 1, wherein the plurality of tubules may be provided in a parallel or mesh configuration.

9. The dynamic blood handing or filtration device according to claim 1, wherein the plurality of tubules may be provided in a linear or nonlinear configuration.

10. A coating for a dynamic blood handling or filtration device, comprising:
a. a specific polyphosphazene coating, the polyphosphazene having the formula: 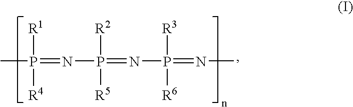
n is 2 to ∞; and
R¹ to R⁶ are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof, or heteroaryl comprising one or more heteroatoms selected from nitrogen, oxygen, sulfur, phosphorus, or a combination thereof.
b. a smooth muscle relaxant active agent.

11. The coating according to claim 10, wherein at least one of R¹ to R⁶ is an alkoxy group substituted with at least one fluorine atom.

12. The coating according to claim 10, wherein R¹ to R⁶ are selected independently from OCH₃, OCH₂CH₃, OCH₂CH₂CH₃, OCF₃, OCH₂CF₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂, OCH₂(CF₂)₆CHF₂, OCH₂(CF₂)₇CHF₂, OCH₂CH═CH₂, OCH₂CH₂CH═CH₂, or any combination thereof.

13. The coating according to claim 10, wherein the polyphosphazene is poly[bis(2,2,2-trifluoroethoxy)]phosphazene or a derivative of poly[bis(2,2,2-trifluoroethoxy)]phosphazene.

14. The coating according to claim 10, wherein the polyphosphazene component is a coating for semipermeable membranous substrate tubule walls.

15. The coating according to claim 10, wherein the smooth muscle relaxant active agent is releasably bonded to the polyphosphazene component.

16. The coating according to claim 10, wherein the smooth muscle relaxant active agent is a compound capable of producing nitric oxide or other bioactive nitrogen compounds upon release from the polyphosphazene component.

17. The coating according to claim 10, wherein the dynamic blood handling or filtration device comprises a plurality of tubules that may be provided in a parallel or mesh configuration.

18. The coating according to claim 10, wherein the dynamic blood handling or filtration device comprises a plurality of tubules that may be provided in a linear or nonlinear configuration.

19. The coating according to claim 10, wherein the coating is applied to a surface of semipermeable membranous substrate tubule walls of a dynamic blood handling or filtration device by dip coating, spray coating, spin coating, brush coating, electrostatic coating, electroplating, or electron beam-physical vapor deposition;

20. A method of providing one or more desired chemical exchanges in a dynamic blood handling or filtration device, comprising:
a. providing a dynamic blood handling or filtration device comprising
(i) a plurality of tubules, at least some of which comprise semipermeable membranous substrate tubule walls;
(ii) a specific polyphosphazene component, the polyphosphazene having the formula: 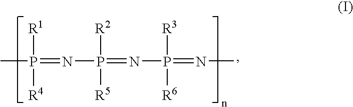
where n is 2 to ∞; and
R¹ to R⁶ are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof, or heteroaryl comprising one or more heteroatoms selected from nitrogen, oxygen, sulfur, phosphorus, or a combination thereof; and
(iii) a smooth muscle relaxant active agent;
b. channeling blood flow within the dynamic blood handling or filtration device; and
c. releasing the smooth muscle relaxant active agent during blood flow.

21. The method according to claim 20, wherein the polyphosphazene is poly[bis(2,2,2-trifluoroethoxy)]phosphazene or a derivative of poly[bis(2,2,2-trifluoroethoxy)]phosphazene.

22. The method according to claim 20, wherein the polyphosphazene component is a coating for the expandable stent structure.

23. The method according to claim 20, wherein the smooth muscle relaxant active agent is releasably bonded to the polyphosphazene component.

24. The method according to claim 20, wherein the smooth muscle relaxant active agent is a compound capable of producing nitric oxide or other bioactive nitrogen compounds upon release from the polyphosphazene component.

25. The method according to claim 20, wherein the semipermeable membranous substrate tubule walls may contact the semipermeable membranous substrate of other tubule walls for the one or more desired chemical exchanges, or wherein the semipermeable membranous substrate tubule walls may contact channeled blood flow or other fluid flow within an interstitial flow space within the dynamic blood handling or filtration device.
